(19) United States
(12) Patent Application Publication
(10) Pub. No.: US 2016/0310593 A1
(43) Pub. Date: Oct. 27, 2016

(54) METHOD FOR CONTROLLED RELEASE WITH FEMTOSECOND LASER PULSES

(71) Applicant: OKINAWA INSTITUTE OF SCIENCE AND TECHNOLOGY SCHOOL CORPORATION, Kunigami-gun, Okinawa (JP)

(72) Inventors: Keshav Moreshwar DANI, Kunigami-gun (JP); Jeffery Russell WICKENS, Kunigami-gun (JP); Takashi NAKANO, Kunigami-gun (JP)

(73) Assignee: OKINAWA INSTITUTE OF SCIENCE AND TECHNOLOGY SCHOOL CORPORATION, Kunigami-gun, Okinawa (JP)

(21) Appl. No.: 15/103,423

(22) PCT Filed: Dec. 11, 2014
(86) PCT No.: PCT/JP2014/083496

§ 371 Date: Jun. 10, 2016

Related U.S. Application Data
(60) Provisional application No. 61/914,750, filed on Dec. 11, 2013.

(51) Int. Cl. A61K 41/00; A61M 5/00; A61N 5/06; A61K 9/127; A61K 31/137

(52) U.S. Cl. CPC A61K 41/0028; A61K 9/1271; A61K 31/137; A61N 5/062; A61M 5/00; A61N 2005/067

(57) ABSTRACT

Provided is a method for controlled release of a chemical substance in vivo with femtosecond laser pulses. The method comprises a step of injecting into the body of a subject a liposome which is filled with the chemical substance and attached to metal nanoparticles. Then, a laser pulse train is applied to the liposome from outside the body with a constant or variable laser intensity, exposure time and time between exposures, thereby releasing a controlled amount of the chemical substance in the body from the liposome on a timescale fast enough to reproduce neural signaling.

### TECHNICAL FIELD

The present invention relates to drug delivery systems for controlled release and pharmaceutical compositions therefore. In particular, the present invention relates to an on-demand, sub-second and repetitive drug delivery system using femtosecond lasers as an external stimulus.

### BACKGROUND ART

Advances in biomaterials and nanotechnology promise the ability to introduce nanoscale devices into living organisms to address, mimic and ultimately control their intrinsic mechanisms. A first application of this concept has been the development of targeted, site specific drug delivery systems activated by external stimuli (see Non-Patent Literature 1). For example, in nano cancer treatments (see Non-Patent Literature 2), the dosage is delivered slowly and continuously over long periods of time at a specific location in the body. Equally important to spatial control is gaining temporal, pulsatile control over the drug delivery system (see Non-Patent Literature 3).

Numerous vital functions of living biological systems occur in a regulated, repeatable manner with natural rhythms of hours (see Non-Patent Literature 4) to milliseconds (see Non-Patent Literature 5). Mimicking these rhythms—that are essential to life chemistry—demands pulsatile, repeatedly-releasing chemical delivery systems with the appropriate temporal profile. Previous attempts have either only achieved temporal control of the pulse profile on the order of hours and days (see Non-Patent Literature 6), or employed a one-time destructive release mechanism by irreversible breakdown of the containing structure.

Liposomal compositions for delivery of a therapeutic or diagnostic agent encapsulated within the liposome have been described. For example, liposomes can be tethered to hollow gold nanoshells (HGNs) and radiating these structures with near-infrared light can trigger the release of the liposomal content (see Non-Patent Literature 7 and Patent Literature 1). However, this construction only allows a one-time, destructive triggering of release of nearly the entire content. In one of their embodiments, where HGNs were directly tethered to the liposomes, irradiation induced a 96% release of the 6-carboxyfluorescein that was stored inside the liposomes, and the radiated HGNs were permanently destroyed, making it impossible to achieve pulsatile, repeatedly-releasing chemical delivery.

Another composition that has been described is a thermally sensitive polymer-particle composite that absorbs electromagnetic radiation and uses the absorbed energy to trigger the delivery of a chemical substance (see Patent Literature 2).

Metal nanoshells are combined with a temperature-sensitive material to provide an implantable or injectable material for modulated drug delivery via external exposure to near-infrared light. Although repetitive release of bovine serum albumin is disclosed in Example 4, the time period between the releases is approximately 20 minutes, which is orders of magnitude slower than is required for a temporal profile that can mimic biological rhythms such as the firing of neuros that occur on microsecond timescales. This is due to the fact that the release mechanism relies on a slow process of swelling and collapsing of the hydrogel matrix in thermal equilibrium.

None of the presently available methods, devices or compositions offers a satisfactory way of attaining robust, repetitive release of chemicals on timescales that are fast enough to reproduce the pulsatile chemical activity of living orgasms.

### PRIOR ART LITERATURE

### Non-Patent Documents

[Non-Patent Literature 1] Ganta, S., Devalapally, H., Shahiwala, A. & Amiji, M. J Control Release, 126, 187-204 (2008).

[Non-Patent Literature 2] Arap, W., Pasqualini, R. & Ruoslahti, E. Science, 279, 377-380 (1998).

[Non-Patent Literature 3] Kikuchi, A. & Okano, T. Adv. Drug Deliv. Rev., 54, 53-77 (2002).

[Non-Patent Literature 4] Welsh, D. K., Logothetis, D. E., Meister, M. & Reppert, S. M. Neuron, 14, 697-706 (1995).

[Non-Patent Literature 5] Buzsáki, G. & Draguhn, A. Science, 304, 1926-1929 (2004).

[Non-Patent Literature 6] LaVan, D. A., McGuire, T. & Langer, R. Nat Biotechnol, 21, 1184-1191 (2003).

[Non-Patent Literature 7] Wu, G. et al. J. Am. Chem. Soc., 130, 8175-8177 (2008).

### Patent Documents

[Patent Literature 1] US 2011/0052671

[Patent Literature 2] WO 01/05586

### OBJECT OF THE INVENTION

An important next step of development is towards sub-second control over the temporal drug-delivery profile in order to mimic faster biological life cycles. A particularly important subsecond biological process is the pulsed release of neurotransmitters and neuromodulators in the brain (Wickens, J. R., et al. Ann N Y Acad Sci, 1104, 192-212, 2007). Chemical synaptic transmission rapidly transmits information between neurons to perform brain functions such as perception and motor control, and learning and memory. Neuromodulators also operate on subsecond timescales to regulate the activity of large swaths of neural tissue (Roitman, M. F., et al. J Neurosci, 24, 1265-1271, 2004). Deficiencies in neurochemical signaling in the brain result in neurological disorders, such as Parkinson's disease. Although replacement therapies have been employed in such disorders, the slow absorption and diffusion of drugs has limited their application to replacement of constant background levels of the neuromodulator (Arbuthnott, G. W. & Wickens, J. R. Trends Neurosci, 30, 62-69, 2007).

Better results can be expected by artificially mimicking the neurochemical signal with the appropriate temporal structure.

An object of the present invention, therefore, is to provide a method allowing sub-second, pulsatile, repeated, on-demand release of chemicals using a nanoscale drug delivery system, fast and robust enough to be capable of mimicking the natural neurotransmitter dynamics in the brain in particular.

### SUMMARY OF THE INVENTION

One aspect of the present invention provides a method for controlled release of a chemical substance in vivo, the method comprising: injecting a liposome into the body of a subject, the liposome being filled with the chemical substance and attached to metal nanoparticles; and applying a laser pulse train to the liposome from outside the body with a constant or variable laser intensity, exposure time and time between exposures, thereby releasing a controlled amount of the chemical in the body from the liposome under a controlled timescale.

In other aspects, systems, devices and compositions for controlled release of a chemical substance in vivo as well as a method for treating a neural disorder are provided by the present invention.

### Effect of the Invention

According to the present invention, a chemical substance can be delivered repeatedly on subsecond timescales by stimulating robust liposome structures filled with the chemical and the delivery time and the chemical concentration can be controlled simply by adjusting the intensity and exposure time of the femtosecond laser pulse train. The ability to mimic and reproduce the subsecond dynamic chemistry of neurotransmitters and neuromodulators in the brain would be a significant step in controlling brain mechanisms, understanding brain behavior, and addressing neurological diseases.

### BRIEF DESCRIPTION OF THE DRAWINGS

FIG. 1: Liposome delivery and measurement system.

(A) Dopamine was encapsulated within the liposome's bimolecular lipid membrane.

Hollow gold nanoshells (HGN) were tethered to the membrane. Femtosecond laser pulse train induces dopamine release from the liposome structures. (B) The released dopamine was measured using fast scan cyclic voltammetry. Triangular voltage pulses were applied to the carbon fiber electrode at 10 Hz. The current response to the voltage pulse showed an oxidation and reduction peak at the respective potentials of dopamine.

FIG. 2: Pulsatile, repeatable dopamine release.

(A) Rapid increase in dopamine concentration stimulated by a one-second laser exposure followed by a decrease due to diffusion. (Inset) Pulsatile release by repeated one-second laser exposures over 100 s of seconds. (B) We observe an initial rapid, and then slow decrease in dopamine released per exposure after multiple laser exposures. This dynamic can be fitted by a bi-exponential curve and is explained by assuming two populations of liposomes with different delivery mechanisms.

FIG. 3: On-demand, repeatable and sub-second drug delivery.

We demonstrate repeated dopamine pulses with arbitrary concentrations and temporal profiles controlled by laser intensity and exposure time respectively. The insets show the linear rise in dopamine concentration during laser exposure, with faster dopamine release rates for higher laser intensities and more prolonged release with longer pulses.

FIG. 4: Electron microscopy images of liposomes attached to carbon fiber.

(A) Carbon fiber to which liposome structures were fixed for repeated measurement. Rectangle denotes the zoomed in region in (B) before liposomes were attached, (C) after liposomes were attached and (D) after laser exposure. One observes a large number of speckles in (C) indicating the attached liposomes and a slightly reduced number in (D) due to losses after laser exposure. A few nominal circles and squares are guides for the eye, with circles as examples of regions where liposomes attach and remain attached after laser exposure (robust population). Squares are examples of regions where liposomes attach but are destroyed after laser exposure (fragile population).

### DETAILED DESCRIPTION OF THE INVENTION

Liposome with Metal Nanoparticles

In one embodiment, the present invention provides a method for controlled release of a chemical substance using a liposome attached to a metal nanoparticle, where the chemical is encapsulated within the liposome. Any chemical substance can be used, as long as they can dissolve into the aqueous solution inside of, or the membrane of, the liposome, which can be a nutrient, or a therapeutic, prophylactic diagnostic or cosmetic agent. The agent can have anti-psychotic anti-proliferative or anti-inflammatory properties or can be neuromodulators or neurotransmitters. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Some other examples include bioactive agents such as antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides, small interfering RNA (siRNA), small hairpin RNA (shRNA), aptamers, ribozymes and retroviral vectors for use in gene therapy.

Examples of neuromodulators and/or neurotransmitters include amines such as dopamine, noradrenaline, and serotonin, amino acids such as GABA, peptides and soluble gases and derivatives thereof. Other examples include acetylcholine, adenosine, and anandamide.

The foregoing substances can also be used in the form of prodrugs or co-drugs thereof. The foregoing substances also include metabolites thereof and/or prodrugs of the metabolites. The foregoing substances are listed by way of example and are by no means to be deemed exhaustive. Other active agents currently available or that may be developed in the future are equally applicable.

The liposome can typically be formed mainly of a phospholipid. Suitable phospholipids include, but are not limited to, L-α-phosphatidylcholine, sphingomyelin, phosphatidylethanolamine, phosphatidylglycerol, phosphatidylserine, phosphatidylinositol, dioleoyl phosphatidylethanolamine and combinations thereof Other lipids can also be employed as long as the packing factor allows formation of a liposome bilayer structure and the structure allows release of the liposomal content when irradiated with laser pulses using the methods of the disclosure. Cholesterol and other substances can be added to adjust the stability of the structure. The outward surface of the liposome bilayer can be modified with polyethylene glycol and similar compounds to avoid detection by the body's immune system, in particular, the reticuloendothelial system, allowing for a longer circulatory life. The liposome may be positively or negatively charged or net neutral.

The composition and size of the liposome can be optimized by considering the factors that affect stability (i.e. the ability for the complex to maintain its structure between release events, survive in storage, survive elimination from the bloodstream and cerebrospinal fluid, and remain impermeable so that drug does not leak out, or the tendency to form clusters), plasticity (i.e. the ability to undergo a release cycle in which the liposome wall temporarily becomes permeable and afterward becomes stable again), and sensitivity (i.e. the intensity of the laser pulse required to cause release). Composition affects the physical and chemical properties. Physical properties affecting stability and plasticity include the diameter of the liposome and the profile of phase transitions of the lipid at storage and body temperature. The physical and mechanical properties of the liposome wall are also dependent on the curvature, and determine the optimal size for mechanical stability. In a preferred embodiment, the liposome comprises a nondestructive structure that transiently becomes permeable when exposed to laser pulses of intensities up to 5 W/cm³, and has a diameter of 10 to 500 nm, more preferably about 200 nm. In one embodiment, the liposome tethered to gold hollow nanoshells was stable in storage for more than 6 months, and when injected in the bloodstream of mice, remained in circulation for more than 3 days.

Metal nanoparticles employed in the disclosure include monodisperse, size-controlled hollow-core nanoshells that absorb strongly in the visible to near infrared spectral range. They can be made of gold, silver or other noble metals.

Conventional galvanic replacement methods can be used as a simple and effective way to prepare a stable, tunable, scalable nanostructure including hollow nanostructures, in which a template metallic nanostructure is contacted with a noble metal salt precursor in an aqueous environment. In this case, the noble metal salt precursor must have a greater standard reduction potential than the template metallic nanostructure. For example, galvanic replacement reactions in which silver templates are replaced with gold can be used to create hollow gold nanospheres (HGNs) with a diameter of 20 to 100 nm. The metallic template core is synthesized using conventional methods and can be a silver particle, which is then mixed with a solution of a metallic salt. Upon mixing, the template core is oxidized to dissolve into the solution as the gold shell is formed as a result of reduction.

Different metallic reagent rations (e.g. silver to gold) produce hollow metallic nanostructures with different sizes and shell thicknesses, which in turn result in absorption peaks at different wavelengths, because absorption occurs due to the surface plasmon resonance effect and the absorption wavelength is determined by the size and geometry (e.g. spheres, cubes, rods, bowls and the like) as well as the type of metal used.

The hollow nanostructure, therefore, can be fine-tuned to absorb at the desired wavelength by adjusting the ratio of the template metal and the noble metal salt, and the absorption wavelength is preferably in the near-infrared region to minimize the attenuation of the light as it passes through tissue.

Examples of noble metal salts that can be used include gold, platinum, silver, palladium, ruthenium, rhodium and iridium. Gold is a particularly effective type of metal, because the surface plasmon resonance for gold occurs at longer wavelengths than many other noble metals and because it poses fewer health risks. Metal salts can be chlorides, acetates, nitrates, or other salts.

Metallic nanostructures can be capped with ligands such as long-chain alkyl thiols. Such ligands or caps include alkanethiols having alkyl chain lengths of about 1 to 30 carbon atoms and polymers such as polyethylene glycol, surfactants, detergents, protein complexes, polypeptides, and other biomolecules such as polysaccharides. Dendrimeric materials, oligonucleotides, fluorescent moieties and radioactive groups can also be used.

Alkanethiols can be modified with chemical moieties and functional groups at various positions. The ligand or the cap can be attached to the metallic nanostructure by various methods including, but not limited to, covalent and electrostatic attachment.

Nanostructures, such as alkylthiol-capped gold nanoparticles, can be dissolved or dispersed in a variety of organic solvents with a wide range of polarity. Other capping agents, such as amines, carboxylic acids, carboxylates and phosphines, can be used to allow the use of virtually any solvent.

These ligands or caps can be used to tether nanostructures to the outside of liposomes using, for example, a thio/PEG-lipid linkage.

The average particle sizes and particle size distributions described herein may be measured using electron microscopy techniques such as SEM or TEM. The references to particle size herein refer to the primary particle size.

Metallic nanostructures can be stabilized against aggregation even in high ionic strength solutions by, coating with, for example, thiolated polyethylene glycol using standard chemistry. The nanostructures stabilized this way can be encapsulated within the lipid bilayers of liposomes. Any number of possible therapeutic or diagnostic agents can be encapsulated within lipid bilayers along with the nanostructures. In another embodiment, nanostructures can also be tethered to the membrane of liposomes through ligand-receptor interactions such as those involving biotin and streptavidin, or with a thiolated polyethylene glycol lipid.

Liposomes may be bound to a solid support such as carbon fiber and be implanted in target locations. Advantages of using such a support include easier administration and targeting, and the ability of the support to store a large amount of liposomal structures and nanoshells. Alternatively, the liposomes may be bound to antibodies that selectively attach to specific sites, cells or molecules, thereby binding the liposomes to the target.

### Laser Pulse Irradiation System

The suspension of liposomes tethered to nanostructures can be irradiated with electromagnetic waves. Upon irradiation, the nanostructure absorbs energy from the radiation and disrupts the liposomal structure or otherwise dissipates the energy in the form of vibrational or thermal energy into the surrounding environment. The electromagnetic radiation used can be generated, for example, with a Ti: Sapphire laser that delivers femtosecond pulses at 800 nm. In other embodiments, lasers generating pulses at different wavelengths in the visible to near-infrared region can be used, typically, at from 650 to 1200 nm. The example below demonstrates that such techniques allow release of dopamine from the liposomes. Dopamine was released within 100 milliseconds after the initiation of the laser irradiation, and the system allowed repeated release of dopamine with precise timing of each pulse. No dopamine was released in control experiments where liposomes without hollow gold nanoshells were used and no dopamine release occurred before irradiation.

The methods of the disclosure can be performed with electromagnetic irradiation of any wavelength to cause the nanostructure to generate heat, or acoustic or pressure waves. Radiation in the visible or infrared range can be used. A laser can be employed to generate irradiation but the disclosure encompasses the use of any radiation source, including sources other than lasers. Alternative radiation sources include, but are not limited to, flash lamps, incandescent sources, radioactive substances and synchrotron radiation.

One advantage of using near-infrared light to trigger release of liposomal content is that near-infrared light can penetrate into tissue, blood, other body fluids and the like, thereby minimizing the attenuation of the light as it passes through the body, and allowing penetration depths of upwards of 10 cm. Sites within the body can be accessed this way, where drug release can be induced upon irradiation in the near-infrared region. Metallic nanostructures, including hollow gold nanoshells, strongly absorb near-infrared light and convert the energy into shock waves, microjets, vibration or heat.

The disclosure demonstrates that absorption by metallic nanostructures of femtosecond pulses in the near-infrared range induces repeated release a soluble model agent, dopamine, which is encapsulated in liposomes. The energy absorbed by the nanostructure leads to production of shock waves or unstable microbubbles, not unlike cavitation bubbles caused by ultrasound. The liposome structure is disrupted by the mechanical and thermal effects of the collapse of microbubbles within milliseconds, and releases the content, as shown, for example, by an increase in the oxidation current of dopamine entrapped in the liposome carrier. The dopamine released from the liposomes appears to be unaffected by this process, and the liposome does not seem to be permanently altered either. Some of the advantages of this radiation-triggered release include (1) localized drug delivery without harming surrounding healthy tissues, (2) no phototoxicity or cutaneous photosensitivity as near-infrared light does not harm tissue and the gold nanoparticles are inert, (3) the targeting of tissue deep inside the body as near infrared light can penetrate deep into tissue, (4) generating high localized concentrations of drugs with both special and temporal control, and (5) repeated release of liposomal content on a timescale that allows mimicking of such biological phenomena as rapid, repeated release of neurotransmitters and neuromodulators, potentially paving ways to treatment of neurological disorders such as Parkinson's disease and Alzheimer's disease. Many other carries and containers than liposomes can be modified by tethering metal nanoparticles to them to realize a system for rapid, repeated release of the encapsulated content on demand upon near-infrared radiation. This system can also be employed to study other fields such as chemical kinetics, membrane chemistry and neuroscience. A variety of excipients can be added to the formulations used in this disclosure. Examples of excipients include chemical stabilizers, buffers, neutral or charged lipids, gases, liquids, oils, and bioactive agents.

The intensity of the radiation is selected based on many considerations such as the degree of attenuation of radiation as it passes through tissue and other media, determined by such factors as the type of tissue targeted and tissue depth. Other considerations include the mechanical, thermal and physical stability of the nanostructure, the liposome, the link that tethers the nanostructure to the liposome, and chemical substances encapsulated inside the liposome. A wide range of intensity can be used as long as radiation at that intensity is sufficient to induce release of liposomal content and does not destroy the liposome or nanostructure instantly. In a preferred embodiment, attenuation effects are taken into account and the intensity of the radiation is adjusted such that the nanostructure is radiated with intensities of up to 5 W/cm², more preferably 2 to 5 W/cm². The intensities herein refer to the intensities at the target site, which may be inside the body, where the nanostructures are located.

The use of a femtosecond laser allows transient, local disruption of the liposomal structure to induce content release. Irradiation typically lasts from 10 femtoseconds to 1 picosecond, preferably 50 to 150 femtoseconds, well before the system comes to thermal equilibrium, followed by a long pause (typically 1 millisecond). Not only does it enable the content to be released instantaneously, but it also ensures that few liposomes or HGNs are destroyed from thermal energy, allowing rapid, repetitive release of a small amount of the content. The time and temporal profile of the laser pulses can be controlled using standard electronics and mechanical shutters known to those in the art. The temporal aspect of the profile includes such factors as intensity, exposure time and time between exposures, which may be constant or varied, but the controlling system can be programmed using standard techniques in the art such that the profile of the pulses encompasses more complex behaviors. The system can also be set up such that irradiation occurs in response to biological or physiological conditions such as electrical or molecular signaling, fluctuations in temperature, concentration of biomolecules, or the onset of diseases such as seizure, ischemia and other disorders.

### Subsecond, Pulsatile and On-Demand Controlled Release

The disclosure provides methods and compositions for remote, targeted, repeated release of a chemical substance from liposomes in vivo, triggered by electromagnetic waves. The liposomes may be located in the bloodstream or other physiological fluids, or within a cell, tissue or orgasm, including humans. The liposomes can also be employed in a biological or chemical experiment. In some embodiments, neurological experiments or assays can be designed using the present disclosure, where chemical substances released from the liposomes can interact with neurons, other cells, or biomolecules to induce or mimic biological signaling. The electromagnetic waves can be infrared radiation generated, for example, by a femtosecond pulsed near-infrared laser. In one aspect, a metal nanoparticle is attached to liposomes by means of ligand-receptor tethering or embedded within the lipid bilayer of the liposomes that encapsulate a drug to be released. The nanostructure absorbs sufficient energy from femtosecond pulses of electromagnetic radiation, typically 10 femtoseconds to 1 picosecond, preferably 50 to 150 femtoseconds, to generate shock waves or heat, or to cause pressure fluctuations or vibrations in the liposome or in the surrounding media, such as water, buffer or physiological fluids. The energy released in these forms mechanically disrupts the membrane of the liposome, triggering rapid release of an encapsulated chemical substance, drug or agent. Spatial and temporal control of content release can be achieved by means of controlled application of radiation. This process can be repeated if necessary, allowing rapid, repeated release of liposomal content. Significant disruption of the membrane of the liposome occurs within 1 millisecond after the initiation of irradiation, and this allows control of liposomal content release with an accuracy up to 1 millisecond.

The nanostructure such as a hollow gold nanoshell strongly absorbs energy from light pulses, and this energy is conducted to the liposomal structure attached to the nanostructure to disrupt the membrane. The energy is also likely to cause unstable microbubbles to form in the surrounding water or other media. These unstable bubbles grow rapidly and undergo violent collapse, producing shock waves or microjets that in turn disrupt the vesicle or liposome carriers. The short length of the pulses ensures that the overall energy input is limited and the bulk sample temperature of the environment only rises by less than 3° C. and as a result irradiation leaves the environment largely intact. The nanoshells themselves are also largely intact after irradiation because the nanoshells are radiated only for an extremely short period of time at a time, thereby allowing the excess energy stored up in the nanoshells to dissipate into the environment when they are not irradiated. The nanoshells can therefore be radiated repeated to induce release of liposomal content. Additionally, no significant increase in temperature means that no significant degradation of the chemical substance encapsulated in the liposome is likely to occur.

The disclosure provides methods and compositions for local, controlled, triggered release of a biomolecule (e.g. dopamine or acetylcholine in the brain or the nervous system), a drug (e.g. an antibiotic at or near the site of inflammation or disease or a chemotherapy drug in or near tumor cells), or other agent (e.g. a nutritious, cosmetic, diagnostic or imagining agent). Controlled, repeated delivery allows slow release of small doses over a long period of time, repeated release at specific times or in response to a physiological condition such as the onset of a seizure, or simulated release of a biomolecule that mimics biological signaling such as release of a neurotransmitter or neuromodulator by neurons. Local delivery allows targeting of specific sites, thereby improving the efficacy of delivery or minimizing the side effects of treatment. In one embodiment, such nanostructures are employed to heat or ablate tumor tissue, in which case, drug release can optionally be induced at the same time. It may be used in place of some treatment methods involving ultrasound cavitation such as the destruction of a kidney stone.

### Treatment Method and Pharmaceutical Composition

The compositions of the disclosure can be delivered to a subject or tissue by intradermal, subcutaneous, intramuscular, intra-arterial, intravenous, and intra-articular injection. For delivery to the brain, a variety of techniques such as the Trojan horse liposome may be employed to transfer the composition across the blood-brain barrier.

For delivery to tumors, the composition can be directly injected into the tumor.

The disclosure further provides methods and compositions for improving the therapeutic or diagnostic efficacy of many agents by, for example, delivering the agents to a specific disease site or other sites of interest, while minimizing their concentration elsewhere in the body. The methods and compositions of the disclosure can also be used to realize targeted delivery by means of antigen-antibody-binding interactions or ligand targeting techniques. The liposomes and other lipid-based drug carriers of the disclosure can sequester toxic drugs within the lipid membrane and offer significant advantages over systemic drug delivery including chemotherapy, by curbing side effects of the drugs at sites that are not targeted and minimizing damage to healthy organs and tissues.

A nanostructure according to the disclosure can be administered alone as a pharmaceutical composition. It can also be administered in combination with a liposomal structure or be formulated with other pharmaceutically acceptable carriers. Suitable pharmaceutical carriers and methods of delivery are known in the art and as described herein.

A pharmaceutical composition of the disclosure can be administered appropriately by preparing the composition with excipients, additives, preservatives, auxiliaries, carriers and components that facilitate triggered release or stabilize the structure. Examples of carriers or auxiliaries include sugar spheres, magnesium carbonate, titanium dioxide, lactose, sucrose, mannitol and other sugars, talc, milk protein, gelatin, starch, vitamins, cellulose, low-substituted hydroxypropyl and its derivatives, animal and vegetable oils, polyethylene glycols and solvents. Intravenous vehicles include fluid such as sterile water and nutrient replenishers. Preservatives include antimicrobial, anti-oxidants, chelating agents, and inert gases. Other pharmaceutically acceptable carriers are known in the art and include aqueous solutions, non-toxic excipients, salts, preservatives, and buffers. The pH and exact concentration of the various components in the pharmaceutical composition are adjusted according to parameters well known in the art. In addition, formulations may be optimized for the desired storage conditions.

Administration of the pharmaceutical compositions according to the disclosure may be local or systemic. By “effective dose” is meant the amount of a liposome or the liposomal contents according to the disclosure for producing a desired or beneficial result to a sufficient degree. Amounts effective for this use will depend on the tissue and tissue depth, the method used for delivery, the wavelength, pulse length, intensity of the radiation used and the like.

Typically, data from in vitro studies on dosage and effects may provide useful guidance in the amounts of the pharmaceutical composition appropriate for administration to a human subject, and animal models may be used to determine effective dosages for specific in vivo techniques. Various considerations are described, e.g., in Langer, Science, 249, 1527, (1990).

The pharmaceutical composition can be administered in a number of ways, such as by subcutaneous or intravenous injection, oral administration, inhalation, transdermal application, or rectal, parenteral or intraperitoneal administration. Depending on the route of administration, the pharmaceutical composition can be coated with a material to protect the pharmaceutical composition, nanostructure, or liposome carrier from the action of macrophages, enzymes, acids, and other natural conditions that may inactivate the pharmaceutical composition or otherwise render the methods of the disclosure less reliable or effective. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use may comprise sterile aqueous solutions (where water soluble) or dispersions and sterile powders for the extemporaneous preparation of sterile injectable solutions or dispersions. Typically, the composition is sterile and fluid to provide easy syringability. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of liposome. The percentage of the compositions and preparations can, of course, be varied and can conveniently be between about 5% to about 80% of the weight of the unit.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Supplementary active compounds can also be incorporated into the compositions.

Application to neuroscience and treatment for neurological disorders

One example of subsecond biological processes is the pulsed release of neurotransmitters and neuromodulators such as dopamine and acetylcholine in the brain (Wickens, J. R. et al. Ann N Y Acad Sci, 1104, 192-212 (200)). Chemical synaptic transmission involves precisely timed pulses of these chemicals and rapidly transmits information between neurons to perform brain functions such as perception and motor control, and learning and memory (Katz, B. Nerve, muscle, and synapse. Mcgraw-Hill Book Co, New York (1966).). Neuromodulators also operate on subsecond timescales to regulate the activity of large swaths of neural tissue (Roitman, M. F. et al. Dopamine operates as a subsecond modulator of food seeking. J Neurosci, 24, 1265-1271 (2004).)

Deficiencies in neurochemical signaling in the brain result in neurological disorders, such as Parkinson's disease. Although replacement therapies for such disorders have been described, the slow absorption and diffusion of drugs has limited their application to replacement of constant background levels of the neuromodulator.

One potential obstacle in applying the present method to treatment of neurological disorders is the presence of the blood-brain barrier, a highly selective permeability barrier separating the brain from the circulatory system. This has hampered many efforts to deliver chemicals into the brain cells. However, liposomes can be used as vehicles to transfer chemicals across the blood-brain barrier (Trojan horse liposomes) (Preparation of Trojan Horse Liposomes (THLs) for Gene Transfer across the blood-Brain Barrier, Cold Spring Harb Protoc (2010)).

The methods of the disclosure can therefore be employed to store a neurotransmitter or a neuromodulator inside the liposomes, and upon irradiation the content can be release in an on-demand, repeated, pulsatile manner to mimic biologically normal processes. This can provide treatment for neurological disorders, including, but not limited to, Parkinson's disease and Alzheimer's disease. In other embodiments, the methods can be used for research on neuroscience in which rapid, repeated biological signaling can be emulated by adjusting the timing of irradiation.

The following non-limiting examples illustrate the various embodiments provided herein. Those skilled in the art will recognize many variations that are within the spirit of the subject matter provided herein and scope of the claims.

Before the present disclosure is described in more detail below as Examples, it should be appreciated that the present invention is not limited to the particular methodology, protocols and reagents described herein as these may vary. It should be also appreciated that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

### EXAMPLES

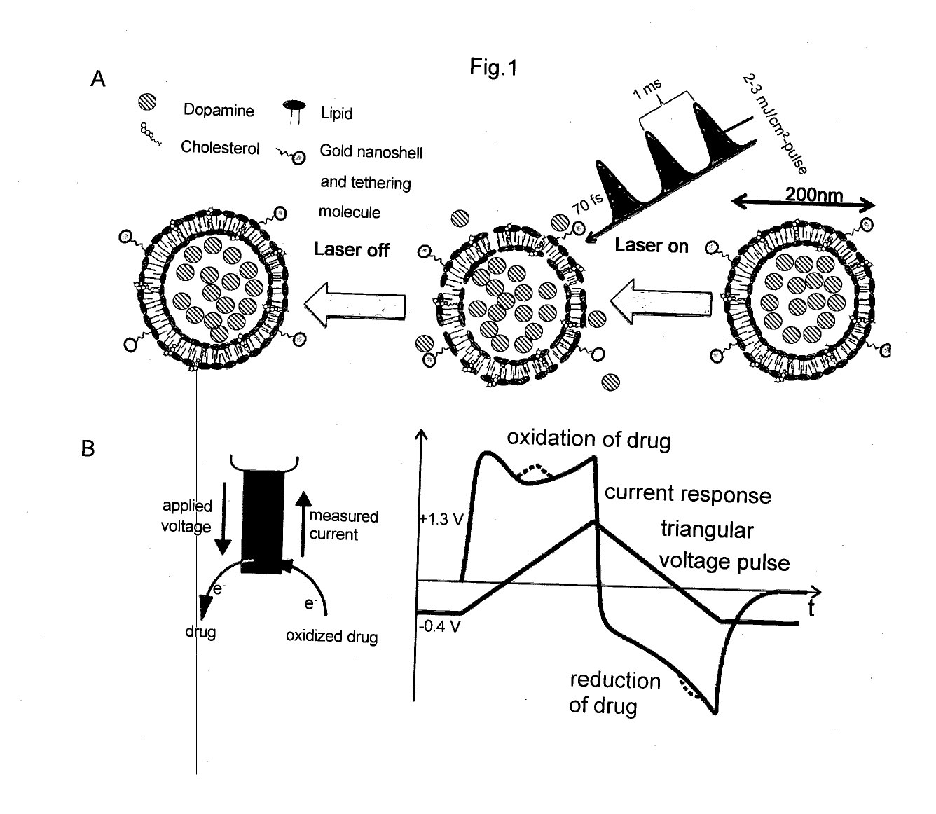

To develop the nanoscale, biocompatible drug delivery system, we prepared liposome structures filled with dopamine, tethered to hollow gold nanoshells (HGN) (Paasonen, L. et al. Journal of Controlled Release, 122, 86-93, 2007) (FIG. 1a). The liposomes were prepared by mixing 1,2-distearoyl-sn-glycero-3-phosphocholine (DSPC), Cholesterol, Sphingomyelin, 1,2-distearoyl-sn-glycero-3-phosphoethanolamine-N-[methoxy(polyethylene glycol)-2000] (DSPC-PEG2000) and DSPE-PEG2000-SH at a molar ratio of 100:5:5:4:3.5. On removal of the solvent (chloroform), a phosphate buffered saline (PBS) containing dopamine was added. The mixture is swirled in a water bath at 50° C. till all lipid materials are suspended and the liposomes are then extruded through a 200 nm polycarbonate membrane allowing us to regulate their size. HGN suspension was then added periodically to the liposomes with a gold-lipid ratio (mg/mmol) of 120:1. This preparation method resulted in stable liposome structures filled with dopamine and tethered to gold nanoparticles. Finally, a carbon fiber of 10 um diameter was dipped in the PBS solution containing the liposome structures for five minutes, thereby allowing a reasonable number of the structures to attach to the fiber. The attachment and consequent immobilization of the liposome structures allowed for their repeatable measurement.

To achieve the release of dopamine from the liposome structures, the carbon fiber with liposomes was submerged in water and illuminated by a train of near-infrared (800 nm) femtosecond pulses. Electron microscope images of the carbon fiber confirmed that the liposomes did not come loose when dipped in tap water, and remain largely intact after repeated laser exposures as discussed below. The femtosecond pulse train had a temporal width of 70 fs per pulse, an adjustable intensity of up to 5 W/cm² and a temporal spacing of 1 ms between pulses (FIG. 1a). An electronically operated mechanical shutter controlled the illumination time of the liposome structures, with typical times of a few hundred miliseconds to a second. The potential mechanisms causing the release of chemicals enclosed within gold tethered liposome structures on exposure to light have been discussed previously (Paasonen, L. et al. Journal of

Controlled Release, 122, 86-93, 2007, and Wu, G. et al. J. Am. Chem. Soc., 130, 8175-8177, 2008).

Thus, by repeated illumination of the same liposome structures with varied laser exposure times, and time between illuminations, we stimulated dopamine delivery with arbitrary temporal profiles.

To measure the release dynamics of dopamine from the light-stimulated liposome structures we used fast scan cyclic voltammetry (FSCV) (Robinson, D. L. et al. Clin Chem, 49, 1763-1773, 2003). In this technique, triangular voltage waveforms (−0.4V to 1.3V and back to −0.4V at 300V/s) are applied to a conducting electrode at a 10 Hz frequency. The electrodes are held at −0.4V between the triangular sweeps (FIG. 1b). We measure the dopamine concentration in solution by recording the current flow during oxidation (reduction) of the dopamine molecule at +0.6V (−0.2V) (Robinson, D. L. et al. Clin Chem, 49, 1763-1773, 2003). In order to sensitively measure the release of dopamine from our liposome structures, we use the same carbon fiber to which the liposome structures were attached as the conducting electrode. This improvisation to the

FSCV technique thus allows us to measure the dopamine release directly at source with high sensitivity. The increased sensitivity, combined with the immobilization of the liposome structures on the carbon fiber, allows us to measure the repeated, on-demand, pulsatile release dynamics of the liposome structures.

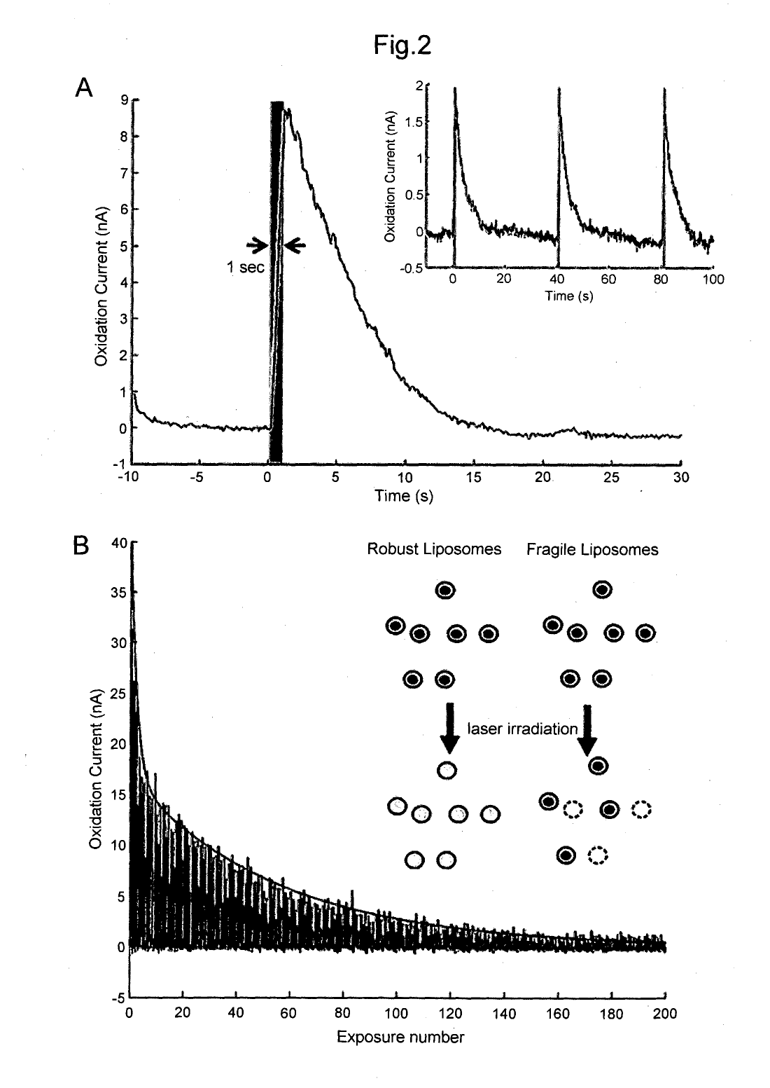

FIG. 2a shows the temporal profile of the dopamine concentration released into the solution due to a single one second, 3 W/cm² illumination with the femtosecond pulse train. We clearly see a rapid and linear rise in the dopamine concentration released while the carbon fiber with the fixed liposome structures is being illuminated. As soon as the laser illumination is shuttered, the dopamine concentration decays exponentially due to diffusion into the solution away from the carbon fiber source. This slow diffusion profile into the solution is expected to vary depending on the solution, dopamine uptake by neighbouring cells and other real-world processes (Cragg, S. J. & Rice, M. E. Trends Neurosci, 27, 270-277, 2004), which are not the focus of this paper. Here we focus on the sub-second controlled rise in the dopamine concentration (i.e. time profile of dopamine delivery) during laser illumination. The inset in FIG. 2a demonstrates pulsatile dopamine delivery achieved via repeated, one-second laser exposures, spaced 40 seconds apart. In each of the dopamine release profiles, we observe the linear and rapid rise in dopamine concentration during the laser exposure, followed by the diffusion of dopamine away from the source.

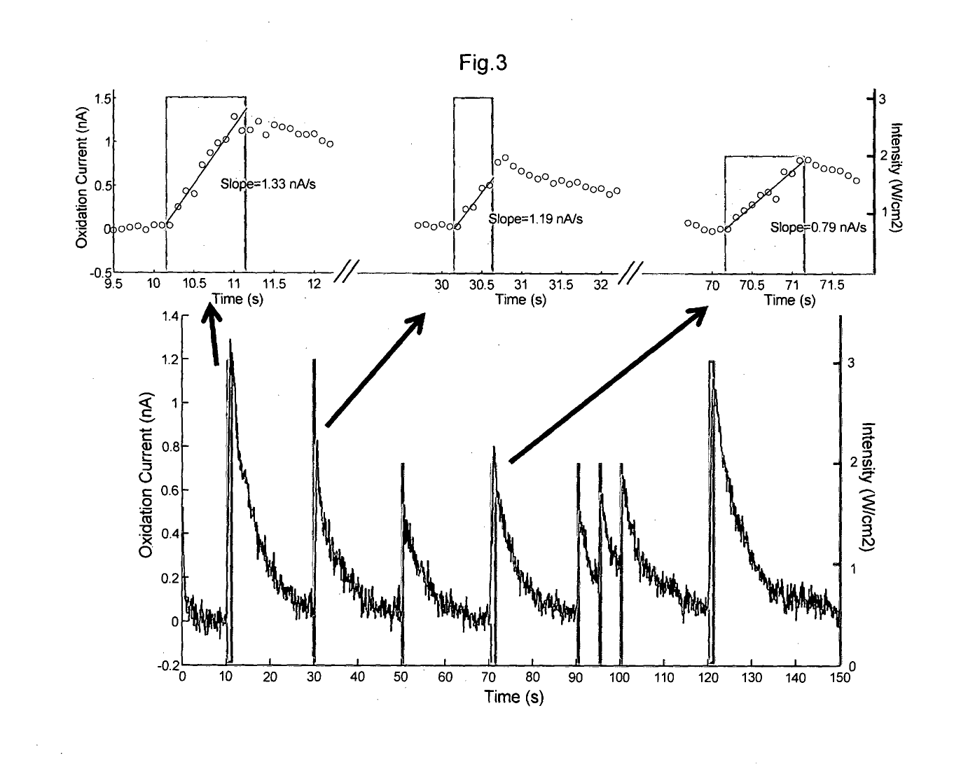

By adjusting the laser intensity, the exposure time, and the time between exposures, we can program an arbitrary pulsatile dopamine release. FIG. 3 demonstrates such an arbitrary pulsatile release profile where the liposome structures were repeatedly illuminated with the femtosecond pulse train using intensities between 2 W/cm² and 3 W/cm², exposure times between 500 ms and 1 second, and the time between exposures ranging from 5 s to 20 s. The insets in FIG. 3 show that the released dopamine increases linearly during the period of illumination, with the rate of release determined by the input laser intensity. Hence it is possible to independently control the temporal profile of the released dopamine (via the exposure time to the laser) and the quantity of the released dopamine (via the intensity of the laser pulse).

In order to further understand the long term behavior and stability of this dopamine delivery system, we measured the peak dopamine concentration released over repeated one-second, 3 W/cm² exposures. The exposures were set 40 seconds apart, thereby allowing the dopamine from the previous release to diffuse away. This time between exposures did not alter the results of the experiments. In FIG. 2b, we plot the peak dopamine concentration released versus the exposure number. The data can be directly fit to a bi-exponential decay, indicating two different processes contributing to the dopamine delivery mechanism—a fast process that lasts only for the first few exposures, and a longer process that lasts for 100 s of exposures.

We mathematically model this behavior by assuming that there are two populations of liposome structures: (i) a ‘fragile’ population with a high probability—α_{f}, of destruction of any one of these liposome structures in a single laser exposure, thereby causing all dopamine within that liposome structure to be released at once (Mackanos, M. A. et al. J Biomed Opt, 14, 044009, 2009); and (ii) a robust population of liposomes which are not destroyed on laser exposure, but laser exposure increases the permeability of their lipid membranes resulting in the fractional release of dopamine molecules into the solution. Such increases in permeability may be due to thermal (Djanashvili, K. et al. Bioorg Med Chem, 19, 1123-1130, 2011) or mechanical (Oerlemans, C. et al. J Control Release, 168, 327-333, 2013) effects from external stimulations as have been proposed previously. With these assumptions, the number of ‘fragile’ liposomes (N_{f}^{k}) surviving after the kₜₕ exposure is given by the exponential decay, where N_{f}⁰ is the initial population of the ‘fragile’ liposomes. The dopamine released in the kₜₕ exposure from these liposomes is simply the number destroyed times the dopamine contained in their internal volume—, where C₀ and V₀ are the concentrations and volumes of each of the as-made liposome structures. For the robust population of liposomes, the number of liposomes doesn't change over time. However, the internal concentration of dopamine continues to deplete due to its partial diffusion into the solution on laser exposure. Assuming that this partial diffusion is merely proportional to the difference in internal and external concentrations (with a proportionality constant of αᵣ), and that the external concentration always goes to zero by the time of the next laser exposure, the dopamine released during the kₜₕ exposure is simply αᵣCᵣ^{k}V₀Nᵣ⁰, where Cᵣ^{k} is the internal dopamine concentration in the robust liposomes during the kₜₕ exposure, and Nᵣ⁰ is the number of robust liposomes. Thus the internal concentration of dopamine follows an exponential decay given by Cᵣ^{k}=C₀e^{-k*α}^{ᵣ}, where C₀ is the as-made initial dopamine concentration. The total dopamine released, given by the sum of the two contributions, thus exhibits a bi-exponential decay with constants α_{f} and αᵣ and magnitudes N_{f} and Nᵣ respectively.

To extract the above parameters from our experiment, we fit the data in FIG. 3b with the expected biexponential decay. This gives us values for α_{f} and αᵣ as 0.29±0.08 (standard error; n=7) and 0.06±0.03 respectively, and the ratio of Nᵣ to N_{f} is 4:1 within experimental error. The large ratio of Nᵣ:N_{f} indicates that only a small percentage of the liposomes are ‘fragile’. Also, the large value of α_{f} indicates that this ‘fragile’ population contributes to the release only in the few initial exposures before they are essentially all destroyed. On the other hand, the ‘robust liposomes constitute a large fraction of the population, and the small a, value demonstrates the possibility of repeated release of dopamine over long periods of time. These robust populations also open the door to future dopamine ‘nano-factories’ within the liposomes (Schroeder, A. et al. Nano Lett., 12, 2685-2689, 2012), which maintain the internal dopamine concentrations and thus eliminate the slow decay component.

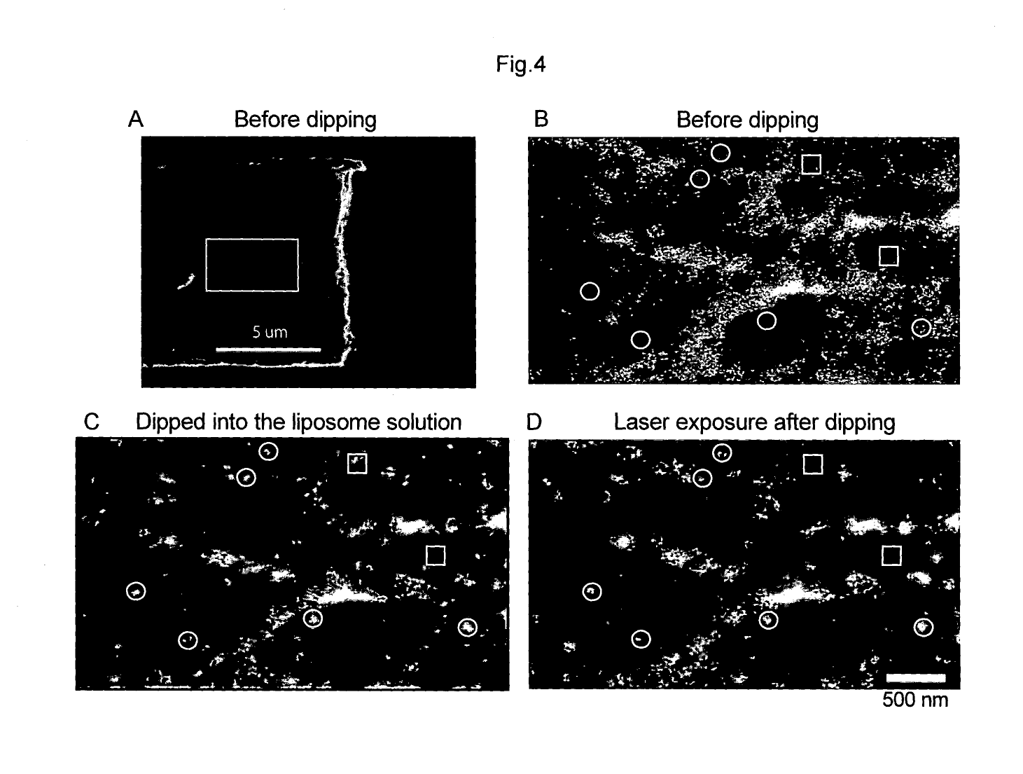

FIG. 4 shows an electron microscope image of the liposome structures before and after laser exposure and provides an independent confirmation of this model. As previously discussed, FIG. 4c shows numerous liposome structures sticking to the carbon fiber before laser exposure, and FIG. 4d images the same region of the carbon fiber after repeated laser exposure. Comparison of these two images indicates that only a small population of the liposome structures have been destroyed (about 20%—consistent with the extracted parameters from our mathematical model), while a large fraction remains after the laser irradiation.

In conclusion, we have demonstrated an on-demand, subsecond, pulsatile, dopamine delivery system using femtosecond lasers as an external stimulus. By varying the laser intensity and exposure time, we can arbitrarily control the concentration and temporal profile of the dopamine delivery. Given the fast timescales on which neural signaling operates, this unprecedented temporal control provides the ability to mimic important neurochemical processes. The technique promises future potential for the delivery of natural and synthetic therapeutic compounds involved in rapid biological signaling; stimulating multiple brain locations simultaneously by combining with recently developed femtosecond techniques to control the size and shape of the stimulated volume (Papagiakoumou, E. et at, Nat Meth, 7, 848-854, 2010) engineering the response of the delivery system to different laser wavelengths to allow for multi-channel operation; and potentially replacing lost functionality due to neural degeneration via ‘neuro-chemical prosthesis’.

What is claimed is:

1. A method for controlled release of a chemical substance in vivo, the method comprising:
injecting a liposome into the body of a subject, the liposome being filled with the chemical substance and attached to metal nanoparticles, and
applying a laser pulse train to the liposome from outside the body with a constant or variable laser intensity, exposure time and time between exposures,
thereby releasing a controlled amount of the chemical substance in the body from the liposome under a controlled timescale.

2. The method according to claim 1, wherein the liposome comprises a nondestructive structure upon exposure of laser pulses with intensities of up to 5 W/cm².

3. The method according to claim 1, wherein the liposome has a diameter of 10 to 500 nm, preferably about 200 nm, and tethered to gold nanoparticles.

4. The method according to claim 3, wherein the gold nanoparticles are hollow gold nanoshells.

5. The method according to claim 1, wherein the liposome is attached to a solid support.

6. The method according to claim 1, wherein the laser pulse train is a train of near-infrared femtosecond pulses with an intensity of less than 5 W/cm², preferably 2 to 3 W/cm².

7. The method according to claim 1, wherein the pulse length is from 10 femtoseconds to 1 picosecond, preferably from 50 to 150 femtoseconds.

8. The method according to claim 1, wherein the chemical substance is a therapeutic agent.

9. The method according to claim 1, wherein the chemical substance is a neurotransmitter, hormone, cytokine or antibody.

10. The method according to claim 1, wherein the laser pulse train is applied in a repeated manner.

11. A system for controlled release of a chemical substance in vivo comprising:
a liposome filled with the chemical substance and attached to metal nanoparticles, and
a laser pulse generator that is capable of irradiating a train of near-infrared laser pulses with a pulse length of 10 femtoseconds to 1 picoseconds, preferably 50 to 150 femtoseconds.

12. A method for treating a neural disorder in a subject in need thereof, the method comprising:
administering to the subject a liposome filled with a neurotransmitter or neuromodulator and attached to metal nanoparticles, the liposome further modified to be transported through the blood-brain barrier, and
applying a laser pulse train to the liposome located in the brain in a repeatable manner with a pulse length of 10 femtoseconds to 1 picoseconds, preferably 50 to 150 femtoseconds.

13. The method according to claim 12, wherein the neuromodulator is dopamine.

14. The method according to claim 13, wherein the dopamine is released with precise timing of pulses with an accuracy of up to 1 millisecond.

15. A pharmaceutical composition comprising a liposome filled with a therapeutic agent and attached to metal nanoparticles for use in a method comprising:
injecting the liposome into the body of a subject, and
applying a laser pulse train to the liposome from outside the body with constant or varied laser intensities, exposure times and time between exposures,
thereby releasing a controlled amount of the therapeutic agent in the body from the liposome under a controlled timescale.
